(19) United States
(12) Patent Application Publication
(10) Pub. No.: US 2015/0150916 A1
(43) Pub. Date: Jun. 4, 2015

(54) INSULIN RESISTANCE-IMPROVING AGENT CONTAINING EGGSHELL MEMBRANE COMPONENT, AND COMPOSITION USING THE SAME

(71) Applicants: THE UNIVERSITY OF TOKYO, TOKYO (JP); ALMADO INC., TOKYO (JP)

(72) Inventors: Yoriko ATOMI, Tokyo (JP); Miho SHIMIZU, Tokyo (JP); Yukio HASEBE, Tokyo (JP)

(21) Appl. No.: 14/553,233

(22) Filed: Nov. 25, 2014

(30) Foreign Application Priority Data
Nov. 29, 2013 (JP) 2013-247189

(51) Int. Cl. A61K 35/57; A23L 1/30

(52) U.S. Cl. CPC A61K 35/57; A23L 1/30; A23V 2002/00

(57) ABSTRACT

To provide an insulin resistance-improving agent, and the application thereof. The insulin resistance-improving agent contains an eggshell membrane component, for example, an eggshell membrane-containing powder or a soluble eggshell membrane component. Further, the eggshell membrane-containing powder to be used is a fine powder, and preferably has a volume average particle size of 6 μm or less, and/or a volume maximum particle size of 20 μm or less, but these particle sizes are not limited to the values.

### CROSS REFERENCE TO PRIOR ART

The present invention claims priority under 35 U.S.C. §119 to Japanese Application No. 2013-247189 filed Nov. 29, 2013, the entire content of which is incorporated herein by reference.

### TECHNICAL FIELD

The entire disclosure of Japanese Patent Application No. 2013-247189, filed Nov. 29, 2013 is expressly incorporated by reference herein.

The present invention relates to an insulin resistance-improving agent containing an eggshell membrane component, for example, a soluble eggshell membrane component (an eggshell membrane hydrolysate, and the like), or an eggshell membrane-containing powder or fine powder, and the application thereof.

### BACKGROUND ART

An eggshell membrane (hereinafter, may also be referred to as “ESM”) is a membrane on the inside of an eggshell of an egg of birds such as an egg of hen, has antimicrobial activity and activity against microorganisms, and protects the developing embryo from infection. The eggshell membrane has a mesh structure composed of a tough fibrous protein and the like, which has type I, type V and type X collagens, glucosamine, desmosine, and hyaluronic acid as the main component. These proteins frequently contain cysteine, are relatively stable against an acid, an alkali, and a protease, and are insoluble in water. The eggshell membrane of an egg of hen has been mostly discarded without being used as a by-product in the food industry, however, it has been known that the eggshell membrane has a function to promote the regeneration of the skin, and particularly a function to promote the generation of type III collagen that is also referred to as fetal collagen. The effectiveness of the eggshell membrane to a living body has begun to attract attention.

The collagen in the skin has known to decrease by aging. The present inventors have reported that type III collagen, decorin, and matrix metalloproteinase-2 (hereinafter, “MMP2”) are significantly higher in the human dermal fibroblasts that are cultured on a water-soluble alkaline-digested form of eggshell membrane (hereinafter, may also be referred to as “ASESM”) bound to an artificial polymer under a condition of the state close to the dermis (a condition under which cells are sparsely present) as compared with those cultured on a collagen-coated dish or a cell culture dish under the same conditions (Non Patent Literature 1: Ohto-Fujita et al., 2011).

Diabetes is a chronic disease that has to be managed for the rest of life, and classified into type 1 diabetes that develops in juveniles who cannot almost make insulin in the pancreas, and type 2 diabetes that is acquired diabetes developed as a result of being triggered by obesity, stress, smoking, lack of exercise, and the like. According to the summary of International Diabetes Federation (http://www.idf.org/diabetesatlas/), the number of people with diabetes in adult (20 to 79 years old) is seems to be around 371 million in 2012 in the world, and the diabetes prevalence rate becomes around 8.3%. The number of people with diabetes continues to increase in the future, and reaches around 552 million, and the diabetes prevalence rate reaches around 9.9% in 2030. In the world ranking of the number of people with diabetes, Japan is the 9th of 7.1 million people in the world, and among them the number of the people who have not diagnosed with diabetes is estimated to be 3.51 million people (the first is China, the second is India, the third is the United States, the fourth is Brazil, the fifth is Russia, the sixth is Mexico, the seventh is Indonesia, and the eighth is Egypt). It has been reported that since Asian people have a thrifty gene that develops diabetes with a little obesity or a little lack of exercise, the risk of developing diabetes is high (Non Patent Literature 2: Diabetes Care, February 2011, Vol. 34, No. 2, 353-357).

Diabetes frequently progresses without exhibiting any symptoms, and the condition of diabetes may frequently progress before the people are aware of the diabetes. Therefore, not only the diet and exercise but also the developments of supplements that prophylactically work are desired. When diabetes progresses, infection easily occurs by the decrease of immune function, cataract easily proceeds, wound becomes difficult to heal as the skin symptom, and the like, and further along with diabetic neuropathy, retinopathy, and nephropathy as a complication, the quality of life (QOL) is reduced. Diabetes is a risk factor for the development of a heart disease and a cerebrovascular disease, and becomes an indirect factor of death. With the data from 1971 to 1980, it was recognized that the life expectancy of a diabetic patient is around 10 years shorter for men, and around 15 years shorter for women, as compared with each life expectancy for ordinary people in japan (Non Patent Literature 3: Sakamoto N, et al., Tohoku J Exp Med 141 (Suppl): 631, 1983).

Patients of hyperglycemia are disabled by impaired the original function of the protein in a living body for non-enzymatically glycosylates. The longer the lifetime of a protein, for example, collagen that is a main component of blood vessels and lens protein crystalline, is affected by the huge effect of the glycation (Non Patent Literature 4: Ulrich P, CeramiA. Recent Prog Horm Res. 2001, 56: 1-21, Review). For example, cataract is caused by aging, and in the condition of high blood sugar, the aging more largely progresses. Arteriosclerosis and microangiopathy also progress according to the similar mechanism. In addition, oxidative stress also increases by free radicals and the like that have been generated by glycation.

Peroxisome proliferator-activated receptor γ (PPARγ) that is a nuclear receptor functions as a mediator when a thiazoline-based (TZD) antidiabetic drug improves the insulin resistance of a type 2 diabetic patient (Non Patent Literature 5: Saltiel A R, Olefsky J M, 1996). Therefore, the PPARγ expression improves insulin sensitivity, and thus has a therapeutic effect on the diabetes. A coactivator, PGC-1α is required for the promotion of the transcription of adipocyte mitochondria uncoupling protein UCP-1 gene by the nuclear receptor of PPARγ-retinoid X receptor (RXR), which contributes to the prevention and improvement of a lifestyle-related disease (Non Patent Literature 6: Puigserver P, et al., 1998; Non Patent Literature 7: Puigserver P, Spiegelman B M, 2003). The UCP expression in adipocytes contributes to the prevention and improvement of a lifestyle-related disease. It is considered that the PGC1α expression promotes the mitochondrial biogenesis, and results in exerting the anti-obesity effect and life lengthening effect, in which Sirt1 is involved as an upstream signal (Non Patent Literature 8: Qiu X., et al., 2010b)). It is known that with the progression of type 2 diabetes inflammatory cytokines are increased in the blood (Non Patent Literature 9: Wellen K E, Hotamisligil G S. J Clin Invest. 2005 May; 115(5): 1111-9. Review). It has been reported that a combination of IL-1 beta and IL-6 are deeply involved in the development of type 2 diabetes (Non Patent Literature 10: Spranger J, Kroke A, Mohlig M, Hoffmann K, Bergmann M M, Ristow M, Boeing H, Pfeiffer A F. Diabetes. 2003 March; 52(3): 812-7), and further, also in the recent meta-analysis, it has been reported that IL-6 and CRP are deeply involved in the development of type 2 diabetes (Non Patent Literature 11: Wang X, Bao W, Liu J, Ouyang Y Y, Wang D, Rong S, Xiao X, Shan Z L, Zhang Y, Yao P, Liu L G. Diabetes Care. 2013 January; 36(1): 166-75. doi: 10.2337/dc12-0702. Review).

It has been reported that an eggshell membrane acts by targeting the following cytokines, and exerts an anti-inflammatory effect: IL-11, IL-1alpha, IL-2, IL-6, NFκB, IL-7, MCP-1, MCP-3, MCP-5, MIP-1 beta, MIP-2, MIP-3 beta, RANTES, TNF alpha, and VEGF (Patent Literature 1).

However, the action mechanism of eggshell membrane components and the method of efficiently enhancing the action of a lifestyle-related disease-associated protein were not sufficiently understood. Further, a substance, which contributes to the physical and mental health by controlling the expression of multiple lifestyle-related disease-associated genes in the skin and the whole body at the same time, has not been known so far.

### CITATION LIST

### Patent Literature

{PTL 1} US patent application No. US20070178170 A1

### Non Patent Literature

{NPL 1} Ohto-Fujita et al., Cell Tissue Res. 2011 July; 345(1): 177-190
{NPL 2} DiabetesCare, February 2011, Vol. 34, No. 2, 353-357
{NPL 3} Sakamoto N, et al.: The features of causes of death in Japanese diabetics during the period 1971-1980. Tohoku J Exp Med 141 (Suppl): 631, 1983
{NPL 4} UlrichP, Cerami A. Protein glycation, diabetes, and aging. Recent Prog HormRes. 2001; 56:1-21. Review
{NPL 5} Saltiel A R, Olefsky J M. Thiazolidinediones in the treatment of insulin resistance and type II diabetes. Diabetes. 1996; 45: 1661-1669. PMid: 20150512
{NPL 6} Puigserver P, Wu Z, Park C W, Graves R, Wright M, Spiegelman B M. A cold-inducible coactivator of nuclear receptors linked to adaptivethermogenesis. Cell. 1998; 92: 829-839
{NPL 7} Puigserver P, Spiegelman B M. Peroxisome proliferator-activated receptor-gamma coactivator 1 alpha (PGC-1 alpha): transcriptional coactivator and metabolic regulator. Endocr Rev. 2003 February; 24(1): 78-90. Review. PubMed PMID: 12588810
{NPL 8} Qiu X., Brown K. V., Moran Y., Chen D. (2010b). Sirtuin regulation in calorie restriction. Biochim.Biophys.Acta 1804, 1576-1583. doi: 10.1016/j.bbapap.2009.09.015
{NPL 9} Wellen K E, Hotamisligil G S. Inflammation, stress, and diabetes. J Clin Invest. 2005 May; 115(5): 1111-9. Review
{NPL 10} Spranger J, Kroke A, Mohlig M, Hoffmann K, Bergmann M M, Ristow M, Boeing H, Pfeiffer A F. Inflammatory cytokines and the risk to develop type 2 diabetes: results of the prospective population-based European Prospective Investigation into Cancer and Nutrition (EPIC)-Potsdam Study. Diabetes. 2003 March; 52 (3): 812-7
{NPL 11} Wang X, Bao W, Liu J, Ouyang Y Y, Wang D, Rong S, Xiao X, Shan Z L, Zhang Y, Yao P, Liu L G. Inflammatory markers and risk of type 2 diabetes: a systematic review and meta-analysis. Diabetes Care. 2013 January; 36(1):166-75. doi: 10.2337/dc12-0702. Review

### SUMMARY OF THE INVENTION

### Technical Problem

The present invention has been made in view of the above-mentioned circumstances. An object of the present invention is to provide an insulin resistance-improving agent that is highly safe, can be routinely used with a simple method, and acts on cells, particularly a co-activation agent of multiple lifestyle-related disease-associated genes; and the application using the same, of a pharmaceutical and/or cosmetic composition, a food (supplement), a food additive, and the like, which are made of a single material, have a multiplex effect, and can maintain and/or improve the living body to a favorable condition.

### Solution to Problem

The present inventors found that an eggshell membrane component acts on the cells of skin and the like, and activates various kinds of lifestyle-related disease-associated genes, and thus have completed the present invention.

Therefore, the object described above is achieved by the following present invention. That is, the insulin resistance-improving agent of the present invention is characterized by containing an eggshell membrane component, in particular, an eggshell membrane-containing powder or a soluble eggshell membrane component (for example, a hydrolysate of eggshell membrane).

In one embodiment of the insulin resistance-improving agent of the present invention, the eggshell membrane-containing powder to be used is fine powder, and preferably the volume average particle size of the eggshell membrane-containing fine powder is 6 μm or less, and/or the volume maximum particle size of the eggshell membrane-containing powder is 20 μm or less.

One embodiment of the insulin resistance-improving agent of the present invention is an expression regulator of one or more genes among PPARγ, UCP, and PGC1α.

In one embodiment of the insulin resistance-improving agent of the present invention, the insulin resistance-improving agent is preferably used in at least any one of the applications of an oral composition, an external composition, a food (supplement), a food additive, a regenerative medical treatment (stem cells, iPS cells, and the like), and a base material.

The composition of the present invention used for a living body, such as a pharmaceutical or cosmetic composition, preferably contains an excipient together with the insulin resistance-improving agent of the present invention. In the case, the composition is preferably an external composition for preventing or improving a lifestyle-related disease, or an oral composition for preventing or improving a lifestyle-related disease. The eggshell membrane component is preferably a soluble eggshell membrane component in an external composition, and an eggshell membrane-containing powder in an oral composition. As one embodiment of the pharmaceutical composition of the present invention, a tablet is preferable.

In another embodiment of the oral composition of the present invention, the eggshell membrane component is preferably contained in a ratio of 5 to 40%. In the embodiment of the external composition, the soluble eggshell membrane component can be contained in a ratio of 1 to 80%, and is preferably contained in a ratio of 1 to 40%.

A food additive of the present invention is characterized by consisting of the insulin resistance-improving agent of the present invention, or containing the insulin resistance-improving agent of the present invention. In addition, in the food of the present invention, it is characterized in that the food additive of the present invention is added.

### Advantageous Effects of the Invention

According to an embodiment of the present invention, an insulin resistance-improving agent containing an eggshell membrane component such as a soluble eggshell membrane or an eggshell membrane-containing powder, and the application thereof, can be provided. According to the insulin resistance-improving agent of the present invention, an extremely safe means in which there is no risk of side effects or the risk is extremely low can be provided in order to adjust the lifestyle-related disease-associated gene expression and to appropriately activate the lifestyle-related disease-associated gene. The insulin resistance-improving agent of the present invention effectively utilizes an eggshell membrane of an egg of hen, which is usually discarded, and can be produced without requiring any complicated processes, therefore, can be readily produced in good yield. The insulin resistance-improving agent is also advantageous from the viewpoint of economy and environmental protection.

Further, the insulin resistance-improving agent of the present invention is made into an appropriate composition depending on the subject to be applied or the purpose, and thus can be widely applied as a functional food, a pharmaceutical preparation such as a prophylactic drug, and a therapeutic drug, and the like. The composition in these embodiments can be routinely easily used by way of application, ingestion, and the like, and can improve the mechanical, physical, and chemical characteristics of water content, elasticity and the like of the skin readily and safely. The insulin resistance-improving agent of the present invention can activate or control the multiple lifestyle-related disease-associated genes in the whole body including the skin at the same time. Therefore, the insulin resistance-improving agent of the present invention can exert the effects of: (1) health maintenance and improvement by the activation of the whole body based on the appropriate promotion of the lifestyle-related disease-associated gene expression; (2) improvement of a slight deviation from the homeostasis range, which is as in “somehow being unsatisfied”; (3) direct improvement of a disease of target cells/tissues; (4) early recovery of a disease by the combination with various kinds of treatments in medical practice using a conditioning effect, and prevention of side effects of a drug such as an anti-cancer agent; (5) buffer function improving the multiple physical deficiencies at a time; (6) prevention and recovery promotion of the injury due to the physical fatigue according to sport; and the like. Further, the insulin resistance-improving agent of the present invention is not stimulative, and can be used in combination with various components in various forms. Therefore, the insulin resistance-improving agent of the present invention can be used in combination with other nutrition (food), and a beauty component in order to obtain further systemic effects, and can also be applied to a composition specialized to the specific effect.

### BRIEF DESCRIPTION OF DRAWINGS

FIG. 1 is a graph showing with the lapse of time the radioactive concentration in blood of a digestion and absorption product of the tritium-labeled eggshell membrane-containing powder that has been orally administered in mice; and

FIG. 2 is an illustration explaining the mechanism in which an eggshell membrane acts on a coactivator PGC-1, and contributes to the improvement and prevention of a lifestyle-related disease (Knutti D, Kralli A. PGC-1, a versatile coactivator. Trends Endocrinol Metab. 2001 October; 12(8): 360-5. Review. PubMed PMID: modification based on Ser. No. 11/551,810).

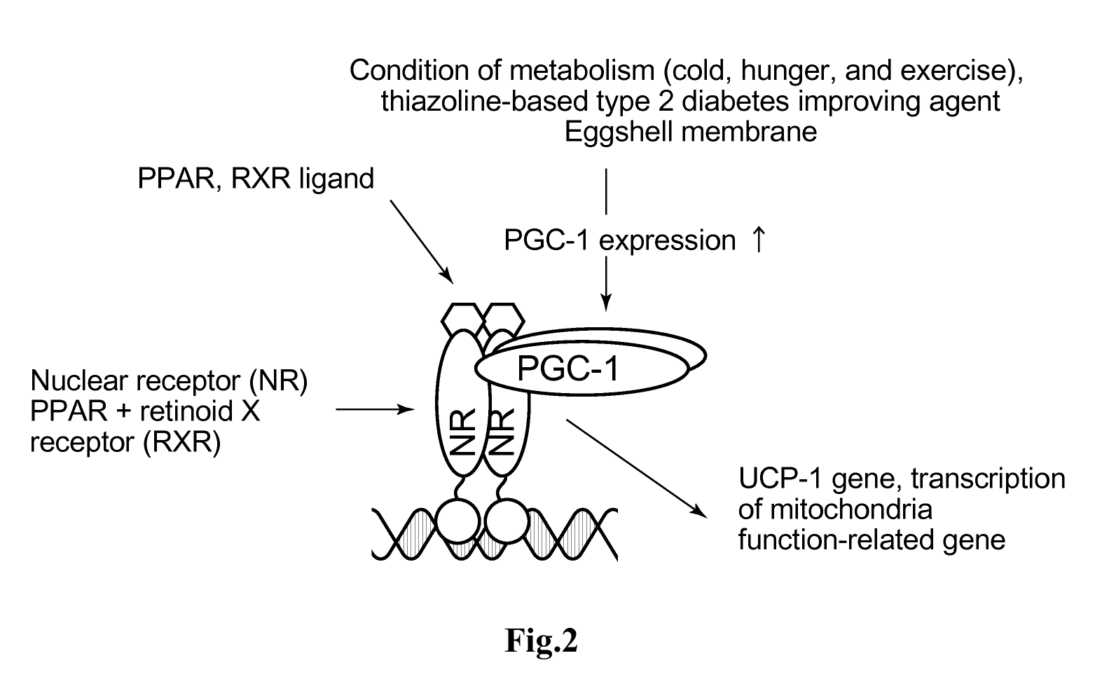

### DESCRIPTION OF EMBODIMENTS

(Insulin Resistance-Improving Agent)

An insulin resistance-improving agent of the present invention uses an eggshell membrane component as an active component. As the eggshell membrane component, any one of an eggshell membrane itself, a product of eggshell membrane, an extract of eggshell membrane, and the like may be used, for example, an eggshell membrane-containing powder, or a soluble eggshell membrane component (a hydrolysate and the like) can be used.

As to the eggshell membrane constituting the eggshell membrane component to be used in the present invention, any one of the membranes (an outer shell membrane and/or an inner shell membrane and/or a limiting membrane), which are obtained from the inside of an eggshell of all of the eggs of a terrestrial oviparous animal, in particular, of eggs of birds, can be used. Among them, in particular, an eggshell membrane of an egg of hen is preferably used in view of the ease of availability, cost, and the like.

(Soluble Eggshell Membrane Component Used in the Present Invention)

The eggshell membrane component used in the present invention can be a soluble component of an eggshell membrane, for example, a decomposition product or extract of an eggshell membrane. The eggshell membrane hydrolysate can be produced in accordance with a known method, for example, a production method of a soluble eggshell membrane, which is characterized in that an eggshell membrane is decomposed in an alkaline water-containing organic solvent, and then the obtained decomposition liquid is neutralized and filtered (Japanese Patent Application Publication (JP-B) No. H06-21047); a production method of a water-soluble eggshell membrane, which is characterized in that an eggshell membrane is treated with a proteinase (JP-B No. H07-110210); a treating method of an eggshell membrane with an anion exchange resin after the hydrolysis in an alkaline water-containing organic solvent (Japanese Patent No. 5179847); and an alkaline hydrolysis method described in U.S. Pat. No. 8,211,477 (Title of Invention: Solubilized protein composition obtained from eggshell membrane) and the modification method thereof.

As to a method other than the alkaline hydrolysis, the soluble eggshell membrane can be produced based on, for example, a picric acid-pepsin treatment method (Takahashi K, Shirai K, Kitamura M, Hattori M. Soluble egg shell membrane protein as a regulating material for collagen matrix reconstruction. Biosci Biotechnol Biochem. 1996 August; 60(8): 1299-302), a method by acid-pepsin hydrolysis (F. Yi, J. Yu, Z. X. Guo, L. X. Zhang, and Q. Li, “Natural bioactive material: a preparation of soluble eggshell membrane protein,” Macromolecular Bioscience, Vol. 3, No. 5, pp. 234-237, 2003; F. Yi, Z. X. Guo, L. X. Zhang, J. Yu, and Q. Li, “Soluble eggshell membrane protein: preparation, characterization and biocompatibility,” Biomaterials, Vol. 25, No. 19, pp. 4591-4599, 2004; Jun Jia, Geng Liu, Jian Yu, and Yuanyuan Duan. 2012. Preparation and characterization of soluble eggshell membrane protein/PLGA electrospun nanofibers for guided tissue regeneration membrane. J. Nanomaterials 2012, Article 25 (January 2012), 1 pages. DOI=10.1155/2012/282736 http://dx.doi.org/10.1155/2012/282736), a method by reduction of S—S bond and trypsin treatment (Kodali V K, Gannon S A, Paramasivam S, Raje S, Polenova T, Thorpe C. A novel disulfide-rich protein motif from avian eggshell membranes. PLoS One. 2011 Mar. 30; 6(3): e18187. doi: 10.1371/journal.pone.0018187), and the like.

The soluble eggshell membrane component may be produced according to these methods by using an eggshell membrane-containing powder described below in place of the eggshell membrane.

The eggshell membrane decomposition product can use the one available on the market. For example, an eggshell membrane hydrolysate of Kewpie (Kewpie Corporation, Tokyo, Japan), trade name “EM PROTEIN-P” can be used.

(Eggshell Membrane-Containing Powder Used in the Present Invention)

The eggshell membrane-containing powder used in the present invention is not particularly limited as long as the powder contains at least an eggshell membrane, however, is preferably an eggshell membrane-containing fine powder and has a volume average particle size of 6 μm or less. Further, the eggshell membrane-containing fine powder used in the present invention preferably has a volume maximum particle size of 20 μm or less. In addition, in the specification of the present application, each of the “volume average particle size” and “volume maximum particle size” of the powder or fine powder means a value measured by using a laser diffraction particle size analyzer (LMS-30, manufactured by SEISHIN ENTERPRISE Co., Ltd.). Herein, the “volume average particle size” means a particle size in 50% of the cumulative value from the small particle size side in the particle size distribution. Further, when the particle size of the eggshell membrane-containing powder or fine powder is measured, a measurement sample in which the eggshell membrane-containing powder or fine powder is dispersed in water by using a surfactant is used. In addition, the “powder” refers to any powder regardless of the size of the particles, and the “fine powder” refers to a powder having a maximum particle size and/or an average particle size of 100 μm or less in general among powders, however, these are not intended to be strictly distinguished.

By the control of the particle size distribution of the eggshell membrane-containing fine powder such that the volume average particle size of the eggshell membrane-containing fine powder is 6 μm or less, or the volume maximum particle size of eggshell membrane-containing fine powder is 20 μm or less, the digestion absorption efficiency and the efficiency of insulin resistance improvement can be further improved as compared with those in the conventional eggshell membrane powder (eggshell membrane powder having a maximum particle size of 100 to 200 μm) obtained by classification treatment with a 70 mesh or a 150 mesh.

The reason why such an effect is obtained is not clear, and it is presumed as follows. In general, the smaller the particle diameter is, the larger the surface area per unit volume of the particle. Therefore, if the particle is constituted of only a substance that is soluble or readily soluble to a digestive juice, the digestion absorption efficiency is improved as the particle diameter is decreased, as a result, the efficiency of insulin resistance improvement is expected to be improved.

However, in the conventional eggshell membrane powder having a maximum particle size of around 100 to 200 μm, and an average particle size of several tens to a hundred and several tens μm order, even if the maximum particle size or the average particle size is changed in these particle size range levels, and the eggshell membrane is more finely ground, the digestion absorption efficiency and the efficiency of insulin resistance improvement are hardly improved. The reason is considered that the eggshell membrane has a strong mesh structure and uses a fibrous protein as the main component, and in the eggshell membrane particles that have been ground in these particle size range levels, the strong mesh structure has been still maintained.

On the other hand, in the eggshell membrane-containing fine powder having a volume average particle size of 6 μm or less or a volume maximum particle size of 20 μm or less, the digestion absorption efficiency and the efficiency of insulin resistance improvement are respectively significantly improved as compared with the conventional eggshell membrane powders. There is speculation that such improvement of the digestion absorption efficiency and the efficiency of insulin resistance improvement is not simply due to the decrease of the particle size, and it is presumed that in the process of micronizing shell membrane, the fibrous strong mesh structure, which is originally possessed by an eggshell membrane, is destroyed in the entire eggshell membrane fine particles, and the entire eggshell membrane fine particles are more easily dissolved to a digestive juice.

Therefore, the powder to be used as an eggshell membrane component in the present invention may have a volume maximum particle size of more than 20 μm, a volume average particle size of more than 6 μm, or a volume maximum particle size of more than 20 μm and a volume average particle size of more than 6 μm, however, from the viewpoint of further improving the digestion absorption efficiency and the efficiency of insulin resistance improvement, an eggshell membrane-containing fine powder having a volume average particle size of 6 μm or less and/or a volume maximum particle size of 20 μm or less is preferable.

In the insulin resistance-improving agent containing an eggshell membrane-containing powder in the present embodiment, at least a powderized or micronized eggshell membrane component is contained, and further a powderized or micronized eggshell calcium component may be contained. In this case, the eggshell membrane-containing powder in the present embodiment is particularly preferably either a form containing only an eggshell membrane component (the first form), or a form containing only an eggshell membrane component and eggshell calcium (the second form). The insulin resistance-improving agent containing the eggshell membrane-containing powder in the first form contains purely only the eggshell membrane component, therefore, can be widely used for various applications of a pharmaceutical composition, in particular, a pharmaceutical composition in a solid form of tablet and the like, a food additive, and the like. Further, in any of the eggshell membrane-containing powder in the first form and the eggshell membrane-containing powder in the second form, it can be accepted that impurity components are contained in the production process and the like. Furthermore, in the insulin resistance-improving agent containing an eggshell membrane-containing powder in the present embodiment, other nutrients and the like may be contained in addition to the eggshell membrane component and the eggshell calcium component.

(Production Method of the Eggshell Membrane-Containing Powder or Fine Powder Used in the Insulin Resistance-Improving Agent of the Present Invention)

In the production of the eggshell membrane-containing powder used in the present invention, a raw material in the state of exfoliated eggshell membrane or a raw material in the state in which an eggshell membrane adheres to the eggshell may be used, and the raw material and the eggshell membrane powder can be used in combination. The method of powderizing such the raw material may be any one of the known methods. An eggshell membrane powder available on the market may be used as the eggshell membrane-containing powder, as the eggshell membrane powder available on the market, for example, trade name “EM powder 300” (manufactured by Kewpie Corporation) can be used. In the case where the eggshell membrane-containing fine powder is produced, an eggshell membrane powder available on the market, or an eggshell membrane powder and eggshell calcium available on the market may be used to be further finely ground until the volume average particle size becomes 6 μm or less and/or the volume maximum particle size becomes 20 μm or less.

The eggshell membrane-containing fine powder used in the present invention can be produced at least through a fine grinding process in which an eggshell membrane-containing raw material is made to collide with one another in a gas to be finely ground. In such the fine grinding process, a so-called jet mill is used. In such the grinding method, frictional heat caused by the contact, collision, or the like of a grinding member with a raw material is hardly generated during the grinding, as compared with a grinding method in which a hard grinding member such as a conventional rotary blade is made to collide with a raw material to grind the raw material, therefore, the damage to a component that is easily modified, deteriorated, and decomposed by heat, such as an amino acid or a protein contained in an eggshell membrane, is small. That is, in the production process, an active component in the eggshell membrane is not easily lost. In addition to this, high pressure gas, not the grinding member, is used in order to grind a raw material, therefore, it is advantageous that impurities derived from a grinding device are not mixed into the eggshell membrane-containing fine powder.

In the fine grinding process, an eggshell membrane-containing raw material is ground preferably until the volume average particle size becomes 40 μm or less, more preferably until the volume average particle size becomes 20 μm or less, and furthermore preferably until the volume average particle size becomes 10 μm or less, by a jet mill. Further, in this case, the eggshell membrane-containing raw material is ground preferably until the volume maximum particle size becomes 20 μm or less. On the other hand, the lower limit of the volume average particle size of the eggshell membrane-containing raw material ground by a jet mill is not particularly limited, however, preferably 4 μm or more, and more preferably 5 μm or more from the practical viewpoint of productivity and the like.

In the eggshell membrane-containing raw material after being ground by a jet mill, if the volume maximum particle size is 20 μm or less and/or, the volume average particle size is 6 μm or less the eggshell membrane-containing raw material can be used as it is as an insulin resistance-improving agent containing an eggshell membrane-containing fine powder of the present invention. On the other hand, in the case where a coarse particle having a particle size of more than 20 μm in a particle size distribution is contained, a classification process in which coarse particles are classified with a sieve having an aperture of 20 μm or less to be removed may further be performed after through the fine grinding process.

Further, in the production method of the eggshell membrane-containing fine powder used in the insulin resistance-improving agent of the present invention, other processes may be performed as needed. For example, the fine grinding process includes the first fine grinding treatment and the second fine grinding treatment, and the raw material powder after the end of the first fine grinding treatment is subjected to a sterilization treatment with high-pressure steam, and then may be subjected to the second fine grinding treatment. In the process in which an eggshell membrane-containing raw material is further ground by a jet mill to be made fine, the antibacterial property of the eggshell membrane easily becomes lower, however, as described in the above, it becomes easy to prevent mold and bacteria from breeding in the eggshell membrane-containing fine powder of the present embodiment as a result of a sterilization treatment.

(Composition Containing an Insulin Resistance-Improving Agent)

A composition of the present invention contains at least one kind of excipient together with the insulin resistance-improving agent of the present invention. Since the insulin resistance-improving agent of the present invention is not stimulative, in the case of using the insulin resistance-improving agent as a composition of a medicine, a cosmetic, or the like, such a composition does not particularly have any limitations in the dosage form, and can be used as any of the oral or external compositions. An external composition of eye drops, nasal drops, ear drops, oral drugs (a mouthwash, and a spray), suppositories (a suppository, an ointment, and an enema) or the like can be prepared in various dosage forms of a liquid preparation, a solid preparation, a semi-solid preparation and the like depending on the intended purpose, by mixing a known component that is usually used. Examples of the composition preferably include, for example, a lotion, an ointment, a gel, a cream, a spray agent, a plaster, and a powder. In the oral administration or the application to be ingested, an oral composition in a form of a tablet, a powder, granules, a capsule, a liquid, and the like are preferred. The oral composition may be a sublingual drug (not only a tablet, but also a sheet such as a wafer paper, and a paste), a jelly, or a drinkable preparation in which fine powders are suspended. In the absorption through the lining of the mouth, the active component directly enters to the heart through the internal jugular vein from a capillary, therefore can avoid the first-pass effect by decomposition and metabolism in the lumen of the gut and by metabolism in the liver, and goes around the whole body at a stretch, thus is advantageous. The various kinds of components and the production method for the production of a composition of a medicine, a cosmetic, or the like in the various dosage forms including the forms described above are known in the field related to the production of a medicine, a cosmetic, or the like, and thus can appropriately be selected by a person skilled in the art as needed. Further, herein the “pharmaceutical composition” is not limited to a human, but includes a pharmaceutical composition for a mammal such as a dog, and a cat, which is kept as a pet or a domestic animal. Furthermore, the “cosmetic composition” includes not only cosmetics but also various kinds of quasi drugs, quasi-drug cosmetics, and the like under the Japanese Pharmaceutical Affairs Law.

In addition, in the present specification and the scope of claims, the “%” means a percentage when the weight or volume of the entire composition is 100%, and in the case where the intended component is in a form of solid (a powder, and the like), the percentage is based on (W/V) or (W/W), and in the case where the intended component is in a form of liquid, the percentage is based on (V/V), unless otherwise noted.

The effective dosage of the pharmaceutical composition for insulin resistance improvement of the present invention varies depending on the disease to be treated or prevented, the type or the degree of the symptom, the condition of the subject to be administered (including the age, the sex, the physical condition, and the like), the dosage form, and the like.

The oral dosage of such the pharmaceutical composition for a human (an adult weighing 60 kg) is preferably 1 mg to 100,000 mg per day in terms of the amount of eggshell membrane component. Specifically, for example, as the effective dosage of the oral pharmaceutical composition of the present invention, an eggshell membrane component can be used in an amount of 18 to 48,000 mg, and further can preferably be used in an amount of 35 mg to 3,500 mg in total per day.

In addition, in the case of an external composition, an external preparation containing an eggshell membrane component in an amount of around 1 to 400 mg/mL (0.1 to 40%) in terms of the amount of eggshell membrane component can be applied once to several times per day, although the amount varies depending on the area, site or the like of the skin to be applied. The application method is not limited to the application, for example, may be spraying in the case where the composition is in a form of liquid, and pasting in the case where the composition is in a form of film, and thus can appropriately be selected.

Since the insulin resistance-improving agent of the present invention is extremely highly safe, and does not have any risk of side effects, there is no problem even if the ingestion amount or the application amount exceeds the range described above, as long as the insulin resistance-improving agent is a composition in which other components are appropriately selected and used.

(External Composition)

A topical application preparation can be prepared in various dosage forms of a liquid preparation, a solid preparation, a semi-solid preparation and the like depending on the intended purpose, by the mixture of the insulin resistance-improving agent of the present invention into a known component that is usually used. In the external composition of the present invention, in addition to the insulin resistance-improving agent of the present invention and an excipient, for example, a cosmetically or pharmaceutically active component, an aroma component (perfume material and the like), a coloring agent and the like can be used. Examples of the other active components include, for example, an antiphlogistic, an anti-inflammatory agent, a melamine production inhibitor, a melanin reducing agent, a decolorizing agent, a melanin drainage accelerator, a cell activator, an antioxidant, an oxidation inhibitor, a keratin dissolving/releasing agent, a sebum inhibitor, a moisturizing agent, an emollient agent, a sebum secretion inhibitor/promoter, an ultraviolet absorbent, an antiperspirant, a blood circulation promoter, a keratin removing/softening agent, a skin-whitening agent, an anti-allergic drug, a steroid hormone, an immunosuppressive agent, and an antibiotic.

For example, by the mixture of the insulin resistance-improving agent of the present invention with one or more component of a hydrocarbon (Vaseline, and the like), a higher fatty acid lower alkyl ester (stearyl alcohol, isopropyl myristate, and the like), an animal oil and fat (lanolin, and the like), polyhydric alcohol (glycerol, and the like), a surfactant (a glycerol fatty acid ester, polyethylene glycol monostearate, and the like), an inorganic salt, a wax, a resin, waster, a preservative (methyl p-hydroxybenzoate, butyl p-hydroxybenzoate, and the like), peptides (acetylhexapeptide-3, palmitoyl pentapeptide-4 (Matrixyl), and the like), acetylated sodium hyaluronate, caprylyl glycol, and the like, a pharmaceutical composition for prevention or improvement of a lifestyle-related disease or for improvement of insulin resistance, or cosmetics can be produced.

The external composition of the present invention preferably contains a moisturizing component and/or a thickening component in the case of an aqueous composition. Examples of the base moisturizing component include, for example, glycerol, diglycerol, polyglycerol, propylene glycol, dipropylene glycol, 1-3 butylene glycol, hexylene glycol, maltitol, mannitol, sorbitol, xylitol, trehalose, sodium pyrrolidone carboxylate, sodium polyglutamate, sodium lactate, sodium polylactate, polyethylene glycol, saccharides, and methylglucoside. Examples of the thickening component include, for example, sodium hyaluronate, sodium dermatan sulfate, dextrin, sodium alginate, carrageenan, xanthan gum, cornstarch, tragacanth gum, casein, polyvinyl alcohol, polyvinylpyrrolidone, methylcellulose, hydroxypropylcellulose, xylan, mannan, galactan, pectin, extensin, gum arabic, pullulan, sodium polyacrylate, carboxyvinyl polymer, and clay minerals. Further, a 2-methacryloyloxyethyl phospholylcholine (MPC) polymer can give an environment close to the skin to fibroblasts, and thus is preferable. Further, there may be a case where 1-3 butylene glycol that is a base moisturizing component is not preferable in relation to allergy, and the 1-3 butylene glycol is preferably appropriately employed in an external composition depending on the symptom and the like.

(Oral Composition)

The insulin resistance-improving agent of the present invention can be used as an oral composition in a form of a tablet, a powder, granules, a capsule, a liquid, or the like. The various kinds of components and the production method for the production of an oral composition in the various dosage forms are known in the field related to the production of a medicine, a cosmetic, or the like, and thus can appropriately be selected by a person skilled in the art as needed.

The oral composition of the present embodiment is preferably contains at least one kind of (1) a health builder (for example, vitamins, β-carotene, royal jelly, and the like), and (2) various kinds of medical components that can be used in combination (for example, an anti-inflammatory agent, and the like), in addition to an excipient.

The type of the vitamin contained in the oral composition of the present embodiment is not particularly limited, and may be any vitamin as long as the vitamin can be ingested by a human or a mammal. For example, examples of the vitamin include fat-soluble vitamins such as vitamin A, vitamin D, vitamin E, vitamin F, and vitamin K, and water-soluble vitamins such as vitamin B, vitamin C, vitamin H, and vitamin L. The tablet of the present embodiment can include one type or two or more types of these vitamins. The content of β-carotene and vitamins can appropriately be determined according to the amount of each vitamin that is suitable for the ingestion by a target such as a human. Further, a vitamin to be contained in the oral composition is not preferably contained according to the recommendations, “as to vitamins and minerals, and an antioxidant supplement, healthy people should not take those as a supplement and should take from the food” from the American Heart Association. However, with the consideration of the need and the like, a vitamin may be contained in some cases.

The oral composition of the present invention is particularly preferably a tablet from the viewpoint that the eggshell membrane is homogeneously contained at a high concentration; the deformation and disintegration are not generated at the time of storage, distribution, taking, or the like; the handleability is excellent; and the taking is easy by mouth. Hereinafter, as an example of the pharmaceutical composition using an insulin resistance-improving agent of the present invention, a tablet will be explained.

The content of the eggshell membrane component in a form of a fine powder, which is contained in a tablet of the present embodiment, is not particularly limited. However, from the viewpoint that the granulation and tableting to particles are smoothly performed; the effect of insulin resistance improvement becomes more excellent when the tablet is orally ingested (taken); the oxygen radicals generated in a living body are decreased, or the scavenging ability of oxygen radicals becomes high; and the like, the eggshell membrane component is preferably contained at a ratio of 5 to 40% by mass, and more preferably 10 to 25% by mass based on the total mass of the tablet.

By the set of the content of the eggshell membrane component to 5% by mass or more, it is not required to ingest a large amount of tablets. On the other hand, by the set of the content of the eggshell membrane component to 40% by mass or less in a tablet, the granulation to particles and the tableting become easy, and the tablet can easily be produced.

In the tablet of the present embodiment, as the various kinds of additives, for example, a binder, a disintegrating agent, a lubricant, other nutrients, and the like can be appropriately added in addition to an excipient in order to form a tablet.

As an excipient for the tablet, at least one kind of modified starch or lactose is preferably used. The content of the excipient is preferably 0.5 to 3 times by mass, and more preferably 1 to 2.5 times by mass, based on the mass of the eggshell membrane component from the viewpoint of the formativeness. Examples of the modified starch include dextrins such as roasted dextrin (white dextrin, yellow dextrin, and the like); oxidized starch (hypochlorous acid-oxidized starch, and the like); and low-viscosity modified starch (acid-dipping starch, enzyme treated starch, and the like). Among them, one kind or two or more kinds can be used. In the case where modified starch (in particular, “Waxy a” and “Pine fiber”) and lactose are used in combination as the excipient, the ratio (mass ratio) of modified starch:lactose is preferably 1:5 to 5:1, and more preferably 1:3 to 3:1.

As the binder, a known binder can appropriately be used, and examples of the binder include, for example, a starch paste, a gum arabic paste, and hydroxypropylcellulose.

As the disintegrator, a known disintegrator can appropriately be used, and, for example, celluloses and the like can be used. In addition, starch has a function as the disintegrator.

As the lubricant, a known lubricant can appropriately be used, and examples of the lubricant include, for example, waxes such as magnesium stearate, and a sucrose fatty acid ester, talc, and vitamin C.

Further, the tablet of the present embodiment particularly preferably contains egg shell calcium as the hardness improving agent, in order to increase the hardness of the tablet, prevent the tablet from being deformed or scratched, improve the handleability of the tablet at the time of packaging, storage, distribution, and the like, and make the ingestion favorable. The egg shell calcium is fine powder prepared by the grinding and drying of shells of the egg of birds such as an egg of hen. In the tablet of the present embodiment, any egg shell calcium can be used as long as the egg shell calcium can be ingested by a human. As the egg shell calcium, for example, egg shell calcium such as trade name “Calhope” manufactured by Kewpie Corporation that is conventionally available on the market, and the egg shell calcium manufactured by Taiyo Kagaku Co. Ltd. can be used as they are. The content of the egg shell calcium contained in a tablet is preferably 5 to 20% by mass, and more preferably 8 to 15% by mass, based on the total mass of the tablet.

The tablet of the present embodiment is preferably covered with a coating for the purpose of the prevention of the components contained in the tablet from deterioration and decomposition, the improvement of the scratch resistance on a surface of the tablet, and the like. The coating can be formed from a coat forming material that is the same as that conventionally used for a coating of a tablet. The coat forming material is not particularly limited, but for example, trade name “Shellac” (Track 30) manufactured by Gifu Shellac Manufacturing Co., Ltd., and the like can be used.

In addition, the tablet of the present embodiment is preferably coated with a sugar coating for ease of oral ingestion, further as needed, may be colored, and may be subjected to a glazing treatment after the coloring.

The size of the tablet in the present embodiment is not particularly limited, and can be appropriately determined, however, in general, the tablet preferably has a circular or elliptical shape with a diameter of around 7 to 10 mm from the viewpoint of the handleability and the ease of the taking.

Further, in the tablet of the present embodiment, for example, the weight per tablet is preferably around 350 to 600 mg, and the eggshell membrane component is preferably contained in an amount of around 18 to 240 mg per tablet, and more preferably 35 to 150 mg per tablet. For example, when the eggshell membrane component is assumed to be contained in an amount of around 18 to 240 mg per tablet of the present embodiment, around 1 to 200 tablets per day can be ingested or administered (18 to 48,000 mg of eggshell membrane component per day in total) for an adult.

The tablet of the present embodiment can be produced with the appropriate use of a known tablet production method, by using a raw material for tableting that contains at least an eggshell membrane-containing fine powder of the present embodiment. Specifically, the tablet of the present embodiment can be produced at least through a naked tablet forming process (tableting process) in which a naked tablet is formed by the tableting by using a raw material for tableting. A granulation process, a protective coating process, a sugar coating process, and the like may be performed in addition to the naked tablet forming process, and further coloring, glazing and the like may also be performed. The tablet of the present embodiment obtained according to the above is shipped by performing sorting, weighing, packaging and the like.

(Food Additive)

The insulin resistance-improving agent of the present invention can be used as a food additive for the addition to a food such as confectionary, a health food, a preserved food, and a processed food, singly or in combination with various kinds of physiologically acceptable components such as other food additives. The food additive of the present invention can be added to various kinds of foods by a method known in the technical field and used for the purpose of the lifestyle-related disease-associated gene activation, or the insulin resistance improvement. For example, as to the application of eggshell membrane for a food, a tablet, confectionary, and the like, which contain the eggshell membrane ground into a powder, have been proposed (Japanese Patent No. 3862600, and Japanese Patent Application Publication No. 2009-165421). As the eggshell membrane powder used in the tablet or confectionary described in these literatures, a food additive containing an insulin resistance-improving agent of the present invention can be used.

Herein the “food” is not limited to a food for a human, but includes a feed for a mammal such as a dog, and a cat, which is kept as a pet or a domestic animal. Further, in the concept of the “food”, a beverage, a so-called supplement or a health food, an enteral nutritive food, a special-use food, a nutritive functional food, a special health food, and the like are included in addition to ordinary foods.

### EXAMPLES

Hereinafter, the present invention will be explained with reference to Examples, but the present invention is not construed as being limited to the following Examples.

### 1. Production of External Preparation Containing Eggshell Membrane Hydrolysate

As the alkaline-hydrolyzed eggshell membrane (hereinafter, referred to as “ASESM”), trade name “EM PROTEIN-P”, which is obtained from Kewpie (Kewpie Corporation, Tokyo, Japan), was used. As to the ASESM, it was found that the relative molecular weight measured by size exclusion chromatography (gel filtration) is around 12 to 14 kDa in the principle parts (Non Patent Literature 1: Ohto-Fujita et al., Cell Tissue Res. 2011 July; 345(1): 177-190).

By using an aqueous solution of 7% (V/V) butylene glycol, 1% (V/V) pentylene glycol, 4% (V/V) glycerol, and 0.2% (V/V) phenoxyethanol as a base, a solution (lotion agent) containing 10% (W/V) ASESM was produced.

### 2. Effect of ASESM External Preparation for Lifestyle-Related Disease-Associated Gene Expression on the Back Part of Hairless Mice

As the animal, a hairless mouse (Hos: HR-1, 6 weeks old, male) was used (a control group: n=9, an ASESM. treated group: n=9). In the ASESM treated group, a 10% (W/V) ASESM solution, which had been produced in the above was externally (locally) applied to the skin on the back part for 10 days (40 μL/one time×2). In the control group, the above-described base solution, which does not contain ASESM, was applied in the same manner.

Quantitative real-time polymerase chain reaction (PCR) analysis was performed in the following manner. A skin sample was collected from each mouse, and ground in liquid nitrogen. After all of the skin tissues were homogenized, the total RNA was isolated by using trade name “TRIzol (registered trademark) Reagent”. The total RNA (200 ng) was applied to cDNA synthesis using trade name “Takara PrimeScript RTR reagent kit”. The real-time PCR method was performed by using trade name “SYBRR Premix Ex Taq™ II (Takara) on Thermal Cycler Dice Real Time System” (Takara). As the primer, a primer, which had been designed to amplify the gene encoding PPARα, PPARγ, and PPARδ, was used. As the internal standard, glyceraldehyde-3-phosphate dehydrogenase (GAPDH) mRNA, which is a housekeeping gene, was amplified in the same manner. The PCR cycle was as follows: the initial denaturation was performed at 95° C. for 30 seconds, and then the amplification of 40 cycles (denaturation at 95° C. for 5 seconds, and annealing and elongation at 60° C. for 1 minute). The primers used are shown together in the following Table 1.

TABLE 1

| SEQ | | Gene | | | | Amplicon |
|---|---|---|---|---|---|---|
| ID | | registered | | | | size |
| NO: | Amplification target | number | Primer name | Sequence (5′ → 3′) | Length | (bp) |
| 1 | Mus musculus peroxisome | NM_011144 | Mm_Ppara-F | TGCTATAATTTGCTGTGGAGATCGG | 25 | 131 |
| 2 | proliferator activated | NM_001113418 | Mm_Ppara-R | GGAAGAGGAAGGTGTCATCTGGAT | 24 | |
| | receptor alpha (Ppara), | | | | | |
| | transcript variant 1 | | | | | |
| 3 | Mus musculus peroxisome | NM_001127330 | Mm/Rn_Pparg-F | CCTTTGGTGACTTTATGGAGCCTAA | 25 | 163 |
| 4 | proliferator activated | NM_011146 | Mm/Rn_Pparg-R | AGGTTGTCTTGGATGTCCTCGAT | 23 | |
| | receptor gamma (Pparg), | | | | | |
| | transcript variant 1 | | | | | |
| 5 | Mus musculus peroxisome | NM_011145 | Mm_Ppard-F | GACCAGAACACACGCTTCCTTC | 22 | 100 |
| 6 | proliferator activator | | Mm_Ppard-R | GCCCCATCACAGCCCATCT | 19 | |
| | receptor delta(Ppard) | | | | | |
| 7 | Mus musculus | NM_008084 | mGAPDH-Left | AGCTTGTCATCAACGGGAAG | 20 | 62 |
| 8 | glyceraldehyde-3- | | mGAPDH-Right | TTTGATGTTAGTGGGGTCTCG | 21 | |
| | phosphate dehydrogenase | | | | | |
| | (Gapdh) | | | | | |
| 9 | Ucp-1 | NM_009463 | Mm_Ucp1-F | TGCCAGGACAGTACCCAAGC | 20 | 104 |
| 10 | | | Mm_Ucp1-R | GAGTCGCAGAAAAGAAGCCACAA | 23 | |
| 11 | Ucp-2 | NM_011671 | Mm_Ucp2-F | AGAACGAGACACCTTTAGAGAAGC | 24 | 182 |
| 12 | | | Mm_Ucp2-R | GCCTTGAAACCAACCATGATTCTG | 24 | |
| 13 | PGC1α | NM_008904 | Mm_Ppargc1a_a-F | AAGCAGAAAGCAATTGAAGAGCG | 23 | 154 |
| 14 | | | Mm_Ppargc1a_a-R | TGAAACCATAGCTGTCTCCATCAT | 24 | |

Each gene expression level was calculated as the ratio of each level of gene expression in the control. The results are shown in Table 2.

TABLE 2

| | Level of gene expression |
|---|---|
| Gene | compared to the control |
| PPAR- α | P = 0.317 ↓ 0.89 times |
| PPAR- γ | P = 0.005 ↑ 1.26 times |
| PPAR- δ | P = 0.685 ↓ 0.92 times |

From these results, it was shown that in the skin on the back part in the ASESM treated group, ASEM acted on cells, as a result, the expression of PARγ, which is a lifestyle-related disease-associated gene, was significantly promoted.

### 3. Production of Eggshell Membrane-Containing Fine Powder

As the sample of eggshell membrane-containing powder, a sample in which trade name “EM powder 300” of Kewpie Corporation had been ground by a jet mill was used. The grinding was performed at an air flow of 1.2 m³/min and a power of 11 kw by using a single track jet mil (FS-4 manufactured by SEISHIN ENTERPRISE Co., Ltd.) as the jet mill, until the volume maximum particle size becomes around 800 meshes (mesh opening is around 20 μm). When the particle size after the grinding was measured by using a laser diffraction particle size analyzer (LMS-30 manufactured by SEISHIN ENTERPRISE Co., Ltd.), the volume maximum particle size was 19.6 μm, and the volume average particle size was 5.8 μm.

### 4. Effect of Eggshell Membrane-Containing Powder Ingestion for Lifestyle-Related Disease-Associated Gene Expression in Mice

A 7-week old male C57BL6/J mouse was made to fast from the previous day. Subsequently 0.5 mg of an experimental supplement (“8φCR 200 mg”, eggshell membrane-containing fine powder produced in the above (800 mesh) 37.50% (75.0 mg), egg shell calcium (Kewpie Corporation) 11.75% (23.5 mg), lactose (Glanbia Foods, Inc.) 43.75% (87.5 mg), a corn protein (KOBAYASHI PERFUMERY CO., LTD.) 5.00% (10.0 mg), and hardened rapeseed oil (Kawaken Fine Chemicals Co., Ltd.) 2.00% (4.0 mg)), in which only eggshell membrane fine powder and egg shell calcium had been contained as the active components; or as a control, 0.5 mg of a control tablet (“9φCR 250 mg”, lactose 93.00% (232.5 mg), a corn protein 5.00% (12.5 mg), hardened rapeseed oil 2.00% (5.0 mg)) (the tablet being powdered in a mortar), in which only an excipient had been contained, was suspended into 100 μL of drug administration jelly for animals (trade name MediGel Sucralose, Japan SLC, Inc.), and the total amount of the resultant suspension was directly administered to the mouse that had been lightly anesthetized with ether through the stomach by using a sonde (n=each 3). Eight hours later, the mouse was dissected, and each gene expression of UCP-1, UCP-2, and PGC1α in a cell in adipose tissue was evaluated by quantitative real-time PCR in the same manner as in the above.

The results are shown in Table 3.

TABLE 3

| | Amount of | | |
|---|---|---|---|
| | eggshell membrane | | |
| | supplement/ | | |
| | per individual | Brown fat | White fat |
| Ucp-1 | 0.5 mg | ↓ 0.91 times | |
| | 1.0 mg | ↑ 1.34 times | |
| Ucp-2 | 0.5 mg | | ↑ 1.63 times |
| | 1.0 mg | | ↑ 1.57 times |
| PGC1 α | 0.5 mg | ↑ 1.37 times | |
| | 1.0 mg | ↑ 3.86 times | |

From these results, it was shown that in the case where eggshell membrane-containing powder had been orally ingested, the expression of UCP, particularly UCP-2, and PGC1α, which are lifestyle-related disease-associated genes, in an adipocyte, was significantly promoted.

### 5. Pharmacokinetics of Eggshell Membrane Component

When a nitrogen-containing compound such as a protein was mixed with lithium carbonate, and then the mixture was irradiated with neutrons, the labeling was performed with the tritium that had been generated by Li⁶(n, α)³H reaction. By using this, the pharmacokinetics when the eggshell membrane-containing powder labeled with tritium had been orally administered to a mouse was examined in the following manner.

<Labeling of Eggshell Membrane>

0.32 g of eggshell membrane-containing powder (“EM powder”, Kewpie Corporation) and 0.65 g of lithium carbonate was thoroughly mixed, and the mixture was encapsulated in a quartz tube under reduced pressure. Subsequently, the mixture was irradiated with neutrons for 20 minutes in Japan Atomic Energy Agency, Nuclear Science Research Institute (JRR4 reactor). The irradiated sample was taken out from the quartz tube, and mixed with water so as to dissolve the unreacted lithium carbonate. Since being insoluble in water, the eggshell membrane powder was collected by filtration. In order to remove the tritium unbound to the eggshell membrane, the filtrate was washed with water until the radioactivity is sufficiently reduced.

<Experimental Animal>

A 6-week old C57BL/6J mouse purchased from Oriental Yeast Co., Ltd. was preliminary raised for around one week (under the environment of a 12-hour light-dark cycle at a temperature of 23±2° C. and a relative humidity of 55±10%,), and then was subjected to an experiment at 7 weeks old. Each mouse was separately raised in a metabolic cage obtained from SUGIYAMA-GEN CO., LTD. (METABOLICA type MM) (86.5 cm²×14.5 cm, and a space of around 2000 cm³), and allowed to freely ingest pelleted food (MF, Oriental Yeast Co., Ltd.) and tap water.

<Administration Method>

The labeled eggshell membrane-containing powder that had been suspended with water was singly forcibly orally administered through the stomach by using a disposable sonde made of plastic to a mouse which had been made to fast for 16 hours before the administration. The administered radioactivity was around 4.5 MBq/kg (122 mCi/kg) body weight, and the dosage was 250 mg/kg body weight.

<Measurement of Radioactivity>

A scintillator was added into the prepared sample for radioactivity measurement, and the measurement of radioactivity was performed by a liquid scintillation counter (Packard, 2200CA). Correction of quenching was performed by an External standard radiation source ratio.

<Measurement of Radioactivity Concentration in Blood>

5 mL of blood was collected from the tail vein 0.25, 0.5, 1, 2, 4, 6, 9, 12, and 24 hours, and 2, 3, 4, 5, 6 days after the administration of labeled eggshell membrane-containing powder. Into this sample, 1 mL of a tissue solubilizer (Soluene-350 (Perkin Elmer)/isopropyl alcohol (1:1)) was added. The resultant mixture was shaken for 3 hours while heating at 50° C., and then into which 500 mL of 30% hydrogen peroxide water was added. Into this sample, 10 mL of a scintillator (Hionic Fluor, Perkin Elmer) was added, and then the radioactivity was measured.

<Excretion of Radioactivity into Urine/Feces>

After the administration of labeling compound, the mouse was placed in a metabolic cage (METABOLICA type MM, SUGIYAMA-GEN CO., LTD.), and the urine/feces were separately collected every day for 6 days after the administration. Part of the collected feces was precisely weighed, and into which 2 mL of a tissue solubilizing agent was added, then the resultant mixture was heated at 50° C. for 3 to 4 hours. Subsequently, 1 mL of isopropanol was added to the mixture, and the resultant mixture was heated at 50° C. for 2 hours. Into this sample, 0.5 mL of 30% hydrogen peroxide water was added, 10 mL of a scintillator (Hionic Fluor, Perkin Elmer) was also added, and then the radioactivity was measured. As to the urine, 5 mL of a scintillator (Ultima Gold LLT) was added into 1 mL of each fraction, and then the radioactivity was measured.

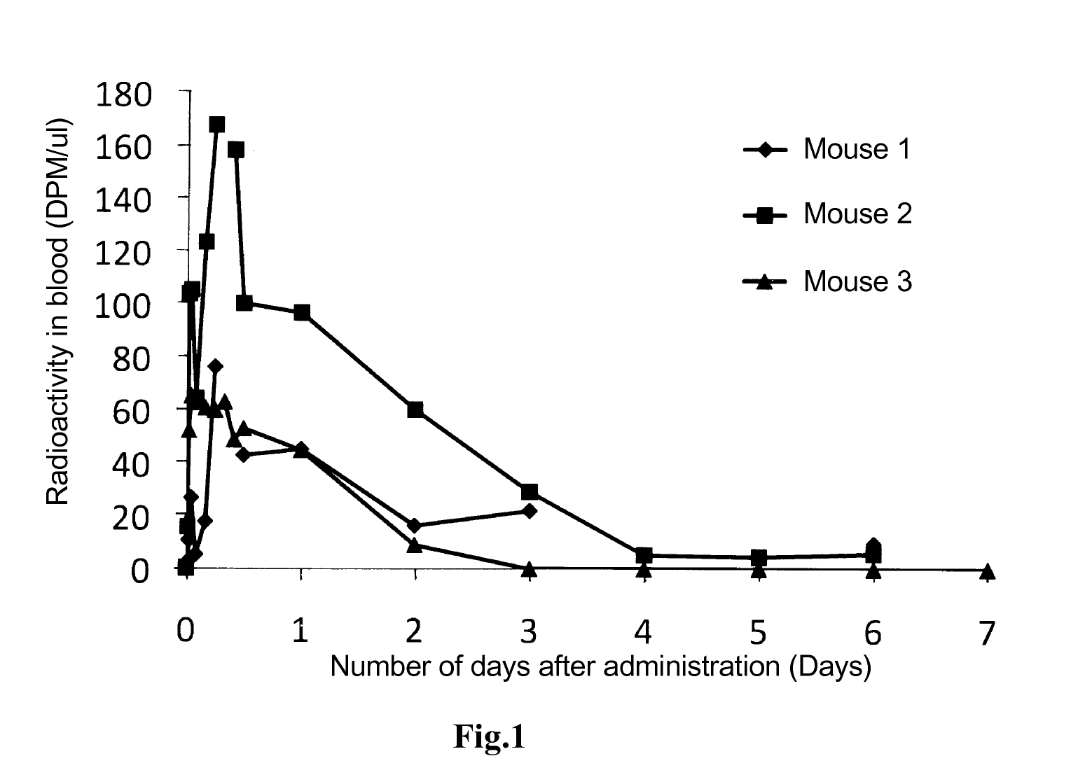

The results of the radioactivity concentration in the blood after eggshell membrane was orally administered to the mouse are shown in FIG. 1. The radioactive concentration in the blood was shown with the lapse of time after the tritium-labeled eggshell membrane was administered. The radioactive concentration in the blood was maximized within 24 hours after the administration, and then decreased to the original radioactivity level in 3 to 4 days.

The results of the radioactivity concentration in urine/feces are shown in Table 4. The radioactivity in urine/feces was 61.5% of the entire dosage in 3 days after the administration.

TABLE 4

| | ³H radioactivity (percentage for dosage (%)) | | |
|---|---|---|---|
| | The first day | The second day | The third day |
| In feces | 14.78 | 33.02 | 11.93 |
| In urine | 0.47 | 0.50 | 0.35 |

<Migration of Radioactivity to all Parts of the Body>

5,568,000 dpm of tritium-labeled eggshell membrane was administered to each individual of 3 mice through the stomach by using a sonde in the same manner as in the above. A part or all parts of the body were excised from each individual 2, 6, and 12 hours after the administration, and weighed. 2 mL of a solubilizer (Soluene-350) was added into each part of the body, and the resultant mixture was incubated at 60° C. for 3 hours. Into this sample, 0.5 mL of 30% hydrogen peroxide water was added, 10 mL of a scintillator (Hionic Fluor) was also added, and the resultant mixture was incubated at room temperature for 1 hour. Subsequently, the radioactivity was measured by a liquid scintillation counter. The results are shown in Table 5 and FIG. 1.

TABLE 5

| | 2 h | 6 h | 12 h |
|---|---|---|---|
| Skin on the back part | 14.39827957 | 26.5335 | 16.06 |
| Large intestine | 3.423908524 | 8.58844221 | 6.5 |
| Small intestine | 4.218064516 | 8.83794393 | 7.12 |
| Pancreas | 12.13833922 | 16.8112288 | 24.69 |
| Duodenum | 3.623255814 | 6.47457627 | 6.57 |
| Intestinal membrane | 2.345011601 | 0.75178571 | 4.33 |
| Kidney | 11.43458498 | 29.500316 | 25.59 |
| Adrenal gland | 5.806666667 | 5.34285714 | 0 |
| Spleen | 6.379558011 | 13.558216 | 9.46 |
| Thymus | 5.085465116 | 13.5094737 | 0.18 |
| Heart | 6.943851508 | 11.6870663 | 5.93 |
| Lung | 2.68342246 | 11.8900826 | 8.89 |
| Liver | 14.24795918 | 28.8658174 | 24.23 |
| Stomach | 27.68076923 | 24.9255708 | 13.85 |
| Bladder | 0 | 4.62395833 | 1.5 |
| Testis | 16.60234375 | 41.5360656 | 33.31 |
| Seminal vesicle | 3.254893617 | 10.1778351 | 21.19 |
| Cerebrum | 5.092879257 | 20.5594406 | 21.05 |
| Gastrocnemius muscle | 5.785743381 | 18.1498305 | 13.87 |
| Visceral fat | 0 | 0 | 0.8 |
| Brown fat | 0 | 0 | 6.79 |
| Soleus muscle | 2.421428571 | 1.84 | 2.73 |
| Hippocampus | 7.248201439 | 24.0738462 | 17.88 |
| White fat on the back part | 6.22173913 | 0.03995215 | 2.21 |

(dpm/mg)

It was revealed that the eggshell membrane component is mostly distributed in various parts of the body, particularly skin, kidney, liver, testis (ovary in a female), brain, (for example, hippocampus). Therefore, it has been expected that the eggshell membrane component shows a promoting effect of lifestyle-related disease-associated gene expression in these parts of the body.

### 6. Production of Pharmaceutical Composition (Tablet)

### (1) Production of Granules for Tableting

20.0 parts by mass of the eggshell membrane-containing fine powder (800 mesh) produced in the above, 10.0 parts by mass of “Waxy a” manufactured by Nisshoku Co., Ltd., 20.0 parts by mass of “Pine Fiber” manufactured by Matsutani Chemical Industry Co., Ltd., 25.9 parts by mass of lactose (manufactured by MEGGLE JAPAN CO., LTD.), 10 parts by mass of egg shell calcium (“Calhope” manufactured by Kewpie Corporation), 5.0 parts by mass of β-carotene, 20.05 parts by mass of vitamin B, 0.05 part by mass of vitamin E, and 2.0 parts by mass of niacin were mixed by using a V type mixer, as a result, the raw material mixture was prepared. Next, to 93.0 parts by mass of this raw material mixture, 15 parts by mass of ethyl alcohol was mixed. The mixture thus obtained was granulated by using a wet granulation apparatus, and then the resultant was dried at a temperature of 50° C. for around 16 hours, as a result, granules for tableting was produced.

### (2) Tableting

Next, to 100 parts by mass of the granules for tableting, vitamin C at a ratio of 9 parts by mass and sucrose fatty acid ester at a ratio of 1 part by mass were mixed. The mixture thus obtained was used to produce a naked tablet with 200 mg per tablet by using a tableting machine.

### (3) Protective Coating

Next, an aqueous solution of “Shellac” manufactured by Gifu Shellac Manufacturing Co., Ltd. was applied on the surface of a naked tablet by using a coating apparatus, and the resultant was dried at a temperature of 40° C. for 2 hours. As a result, a tablet on which protective coating was performed (protectively coated tablet) was obtained.

### (4) Sugar Coating

On the surface of the protectively coated tablet that had been thoroughly dried, paste A for sugar coating (a paste in which 70 parts by mass of granulated sugar, 3 parts by mass of gum arabic, 4 parts by mass of gelatin, 3 parts by mass of egg shell calcium, and 65 parts by mass of water had been mixed) was coated by using a sugar coating apparatus, and then the resultant was dried at a temperature of around 40° C. for around 4 hours. After that, paste B for sugar coating in which paste A for sugar coating is diluted with water was prepared. Further, on the surface of the tablet that had been subjected to coating treatment and drying treatment with paste A for sugar coating, paste B for sugar coating was coated by using a sugar coating apparatus, and then the resultant was dried at a temperature of around 40° C. for around 4 hours. As a result, a tablet that had been coated with a paste for sugar coating (sugar-coated tablet) was obtained.

### (5) Coloring

On the surface of a sugar-coated tablet, a coloring liquid containing “SR Red K3” manufactured by San-Ei Gen Inc. was applied, and then the resultant was dried at 40° C. to 50° C. for 4 hours, as a result, a red-colored tablet (colored tablet) was produced.

### (6) Glazing

On the surface of a colored tablet, glazing was performed by using carnauba wax. The mass per tablet of the table that had been obtained according the above was 400 mg, and the eggshell membrane component was contained in an amount of around 40 mg per tablet.

### (7) Sorting-Weighing-Packaging

The tablets that had been subjected to glazing treatment were sorted so that defective tablets are eliminated. The remained tablets were weighed after the product inspection, and packaged in a double bag in which a desiccant had been contained. Further, the tablet has sufficient hardness and a sufficient shape retaining property, and is excellent in the handleability without having any deformation and disintegration during the sorting, the inspecting, and the packaging.

As above, while certain embodiments and Examples of the present invention have been described, the present invention may be changed in various ways without departing from the spirit thereof.

1. An insulin resistance-improving agent, comprising:
an eggshell membrane component.

2. The insulin resistance-improving agent according to claim 1,
wherein the eggshell membrane component is a soluble eggshell membrane component or an eggshell membrane-containing powder.

3. The insulin resistance-improving agent according to claim 1,
wherein expression of one or more genes among PPARγ, UCP, and PGC1α is enhanced.

4. The insulin resistance-improving agent according to claim 3,
wherein expression of two or more genes among PPARγ, UCP, and PGC1α is enhanced.

5. An external composition for preventing or improving a lifestyle-related disease, comprising:
the insulin resistance-improving agent according to claim 1, and
an excipient.

6. The external composition according to claim 5,
wherein the eggshell membrane component is a soluble eggshell membrane component.

7. An oral composition for preventing or improving a lifestyle-related disease, comprising:
the insulin resistance-improving agent according to claim 1, and
an excipient.

8. The oral composition according to claim 7,
wherein the eggshell membrane component is an eggshell membrane-containing powder.

9. A food additive, comprising:
an insulin resistance-improving agent comprising:
an eggshell membrane component.

10. A food,
wherein a food additive comprising:
an insulin resistance-improving agent is added comprising:
an eggshell membrane component.


SEQUENCE LISTING (14 sequences)

SEQ ID NO: 1 - DNA, 25 bp, Mus musculus
tgctataatttgctgtggagatcgg

SEQ ID NO: 2 - DNA, 24 bp, Mus musculus
ggaagaggaaggtgtcatctggat

SEQ ID NO: 3 - DNA, 25 bp, Mus musculus
cctttggtgactttatggagcctaa

SEQ ID NO: 4 - DNA, 23 bp, Mus musculus
aggttgtcttggatgtcctcgat

SEQ ID NO: 5 - DNA, 22 bp, Mus musculus
gaccagaacacacgcttccttc

SEQ ID NO: 6 - DNA, 19 bp, Mus musculus
gccccatcacagcccatct

SEQ ID NO: 7 - DNA, 20 bp, Mus musculus
agcttgtcatcaacgggaag

SEQ ID NO: 8 - DNA, 21 bp, Mus musculus
tttgatgttagtggggtctcg

SEQ ID NO: 9 - DNA, 20 bp, Mus musculus
tgccaggacagtacccaagc

SEQ ID NO: 10 - DNA, 23 bp, Mus musculus
gagtcgcagaaaagaagccacaa

SEQ ID NO: 11 - DNA, 24 bp, Mus musculus
agaacgagacacctttagagaagc

SEQ ID NO: 12 - DNA, 24 bp, Mus musculus
gccttgaaaccaaccatgattctg

SEQ ID NO: 13 - DNA, 23 bp, Mus musculus
aagcagaaagcaattgaagagcg

SEQ ID NO: 14 - DNA, 24 bp, Mus musculus
tgaaaccatagctgtctccatcat